(19) United States
(12) Patent Application Publication
(10) Pub. No.: US 2023/0305088 A1
(43) Pub. Date: Sep. 28, 2023

(54) LIGHTWEIGHT MAGNET ARRAYS FOR MRI APPLICATIONS

(71) Applicant: EPSITAU LTD, Tel aviv (IL)

(72) Inventor: Noam Haham Hay, Tel Aviv (IL)

(73) Assignee: EPSITAU LTD., Tel Aviv (IL)

(21) Appl. No.: 18/021,432

(22) PCT Filed: Aug. 15, 2021
(86) PCT No.: PCT/IB2021/057513

§ 371 Date: Feb. 15, 2023

Related U.S. Application Data
(60) Provisional application No. 63/066,286, filed on Aug. 16, 2020.

(51) Int. Cl. G01R 33/383; G01R 33/421; G01R 33/38

(52) U.S. Cl. CPC G01R 33/383; G01R 33/421; G01R 33/3802; A61B 5/055

(57) ABSTRACT

A magnet array includes multiple magnet elements made of a permanent magnet material and a frame. The multiple magnet elements are dispersed around a longitudinal axis. At least some of the magnet elements form a ring which is coaxial with the longitudinal axis. The ring comprises at least one magnet element which possesses a cylindrical symmetry around its own axis of symmetry, wherein the axis of symmetry has a component in a direction tangential to the peripheral shape of the ring. The magnet elements are configured to produce a uniform magnetic field inside an inner predefined volume. The frame is configured to fixedly hold the multiple magnet elements in place.

### CROSS-REFERENCE TO RELATED APPLICATIONS

This application claims the benefit of U.S. Provisional Patent Application 63/066,286, filed Aug. 16, 2020, whose disclosures are incorporated herein by reference.

### FIELD OF THE INVENTION

The present invention relates generally to magnet assemblies, and particularly to lightweight magnet assemblies comprising permanent magnets and design methods thereof, and to the use of such magnet assemblies for MRI systems.

### BACKGROUND OF THE INVENTION

Designs of permanent magnet arrays aiming at achieving a strong and uniform magnetic field have been previously reported in the patent literature. For example, U.S. Pat. No. 7,423,431 describes a permanent magnet assembly for an imaging apparatus having a permanent magnet body having a first surface and a stepped second surface which is adapted to face an imaging volume of the imaging apparatus, wherein the stepped second surface contains at least four steps.

As another example, U.S. Pat. No. 6,411,187 describes adjustable hybrid magnetic apparatus for use in medical and other applications includes an electromagnet flux generator for generating a first magnetic field in an imaging volume, and permanent magnet assemblies for generating a second magnetic field superimposed on the first magnetic field for providing a substantially homogenous magnetic field having improved magnitude within the imaging volume. The permanent magnet assemblies may include a plurality of annular or disc like concentric magnets spaced-apart along their axis of symmetry. The hybrid magnetic apparatus may include a high magnetic permeability yoke for increasing the intensity of the magnetic field in the imaging volume of the hybrid magnetic apparatus.

U.S. Pat. No. 10,018,694 describes a magnet assembly for a magnetic resonance imaging (MRI) instrument, the magnet assembly comprising a plurality of magnet segments that are arranged in two or more rings such that the magnet segments are evenly spaced apart from adjacent magnet segments in the same ring, and spaced apart from magnet segments in adjacent rings. According to an embodiment, a plurality of magnet segments is arranged in two or more rings with the magnetization directions of at least some of the magnet segments being unaligned with a plane defined by their respective ring, to provide greater control over the resulting magnetic field profile.

U.S. Pat. No. 5,900,793 describes assemblies consisting of a plurality of annular concentric magnets spaced-apart along their axis of symmetry, and a method for constructing such assemblies using equiangular segments that are permanently magnetized.

### SUMMARY OF THE INVENTION

An embodiment of the present invention provides a magnet array including multiple magnet elements made of a permanent magnet material and a frame. The multiple magnet elements are positioned along a longitudinal axis which passes through a predefined inner volume. At least one group of the magnet elements forms a ring and at least one magnet element of the ring possesses cylindrical symmetry with respect to its own axis of symmetry, wherein the axis of symmetry of the magnet element has a finite component in a direction tangential to the peripheral shape of the ring. The multiple magnet elements are configured to jointly generate a magnetic field of at least a given level of uniformity inside the inner imaging volume. The frame is configured to fixedly hold the multiple magnet rings in place.

In some embodiments the permanent magnet elements form multiple magnet rings coaxial with the longitudinal axis.

In some embodiments, the multiple magnet elements are configured to jointly minimize a fringe field outside the magnet array.

In an embodiment, each magnet ring has a rotational symmetry with respect to an in-plane rotation of the ring around the longitudinal axis.

In another embodiment, at least some of the elements encircle the predefined inner volume of the MRI system, wherein the magnet elements are divided into (i) a first assembly characterized by a first minimal inner radius that is smallest among distances of the magnet elements of the first assembly to the longitudinal axis, and (ii) a second assembly positioned alongside the first assembly along the longitudinal axis and characterized by a second minimal inner radius that is smallest among the distances of the magnet elements of the second assembly to the longitudinal axis, wherein the first minimal inner radius of the first assembly is larger than the second minimal inner radius of the second assembly, wherein a center of the imaging volume is located outside the second assembly.

In an embodiment, the second assembly is positioned along the longitudinal axis on one side of the imaging volume and at least one of the magnet elements in the first assembly is located along the longitudinal axis on a second side of the imaging volume.

In some embodiments, the magnet elements are arranged with reflectional asymmetry with respect to the longitudinal axis.

In some embodiments, the inner volume is an ellipsoid of revolution around the longitudinal axis.

In some embodiments, each of the magnet rings has a shape including one of an ellipse, a circle, and a polygon.

There is additionally provided, in accordance with an embodiment of the present invention, a method for producing a magnet array, the method including positioning multiple magnet elements made of a permanent magnet material around a longitudinal axis which passes through an inner predefined imaging volume of the MRI system, wherein at least one group of magnet elements forms a ring coaxial with the longitudinal axis, wherein at least one magnet element of the ring possesses cylindrical symmetry with respect to its axis of symmetry, wherein the axis of symmetry of the magnet element has a finite component in a direction tangential to the peripheral shape of the ring. The multiple magnet elements are configured to jointly generate a magnetic field of at least a given level of uniformity inside the inner imaging volume; and a frame, which is configured to fixedly hold the multiple magnet rings in place.

The present invention will be more fully understood from the following detailed description of the embodiments thereof, taken together with the drawings in which:

### BRIEF DESCRIPTION OF THE DRAWINGS

FIG. 1 is a perspective view of an asymmetric magnet array comprising a first magnet assembly and a second magnet assembly, according to an embodiment of the present invention;

FIGS. 2A and 2B-2D are a perspective view of an asymmetric magnet array, and plots of magnetic field lines generated separately and jointly by the assemblies, respectively, according to another embodiment of the present invention;

FIG. 3 is a perspective view of a segmented magnet ring, which may be any one of the rings in the magnet arrays of FIGS. 1 and 2, according to an embodiment of the present invention;

FIG. 4 is a perspective drawing of a magnet array of phase-dissimilar mixed-phase magnet rings (MPMRs), according to an embodiment of the present invention;

FIG. 5 is a plot of magnetic field lines generated by the magnet array of FIG. 4, according to an embodiment of the present invention;

FIG. 6 is a perspective view of a mixed-phase magnet ring (MPMR), which may be any one of the rings in the magnet array of FIG. 4, according to an embodiment of the present invention;

FIG. 7 is a perspective drawing of an asymmetric magnet array comprising three theta magnetic rings, according to an embodiment of the present invention;

FIG. 8 is a plot of magnetic field lines generated by the magnet array of FIG. 4, according to an embodiment of the present invention;

FIG. 9 is a perspective view of theta magnet rings, which may be any one of the rings in the magnet array FIG. 7, according to embodiments of the present invention;

FIG. 10 is a perspective view of exemplary optional permanent magnet segments which possess cylindrical symmetry; (a) a cylinder, (b) a sphere, (c) an ellipsoid, (d) a general shape;

FIG. 11 is a perspective view of exemplary rotationally symmetric rings which could be MPMRs, theta rings, or segmented rings, wherein the rings are composed of segments having a cylindrically symmetric shape according to embodiments of the present invention;

FIG. 12A-12B are schematic side views of an exemplary ambulance combined with magnetic shielding and an MRI device, according to an embodiment of present invention;

FIG. 13 is a schematic top cross-sectional view of an exemplary ambulance with a magnetically shielded rear door according to an embodiment of the present invention, and;

FIG. 14 is a schematic top cross-sectional view of an exemplary ambulance having a magnetically shielded rear door which is composed of two moving parts, according to an embodiment of the present invention.

### DETAILED DESCRIPTION OF EMBODIMENTS

### Overview

Magnetic fields that are strong and uniform are needed in a wide variety of disciplines, spanning medicine, aerospace, electronics, and automotive industries. As an example, magnets used in Magnetic Resonance Imaging (MRI) of the human brain typically provide a magnetic field with a strength of 0.1 to 3 Tesla, which is uniform to several parts per million (ppm) inside an imaging volume of approximately 3000 cubic centimeters, e.g. the interior of a sphere of radius 9 cm. However, such magnets have limited applications due to their considerable size and weight. Moreover, in general with magnet designs, there is a severely limiting trade-off between weight, magnetic field uniformity, and a size of a volume inside which a given uniformity can be achieved. Embodiments of the present invention that are described hereinafter provide lightweight permanent magnet arrays that generate strong and uniform magnetic fields (e.g., in the range of 0.1 to 1 Tesla). Some of the disclosed magnet arrays are configured for emergency-care brain mobile MRI systems, such as a head MRI system inside an ambulance. Generally, however, the disclosed techniques can be applied in any other suitable system.

In the description herein, using a cylindrical reference frame consisting of longitudinal (Z), radial (r), and azimuthal (θ) coordinates, an inner volume is defined as a volume of an ellipsoid of revolution around the longitudinal axis. Examples of an inner volume are a prolate having its long axis along the longitudinal axis, and an oblate having its short axis along the longitudinal axis. A lateral plane is further defined as any r-θ plane (i.e., a plane orthogonal to the longitudinal z-axis). A particular definition of an inner volume is an imaging volume of an MRI system inside which the magnetic field has at least a given level of uniformity.

In some embodiments of the present invention, a magnet array is provided that comprises a frame, which is configured to hold, fixed in place, multiple magnet elements made of a permanent magnet material and dispersed around a central longitudinal axis at different positions along the axis, wherein at least some of the magnet elements form a ring coaxial with the longitudinal axis. In the present description a frame is defined by its mechanical capability to hold the rings in place, and which can be made in various ways, for example, using a yoke or by embedding the rings in a surrounding material (e.g., in epoxy).

In some embodiments at least one ring encircling an area contained in an inner volume through which the longitudinal axis passes (i.e., the ring intersects the inner volume).

In some embodiments the magnet elements form multiple magnet rings coaxial with the longitudinal axis.

In some embodiments some or all rings are made of segments, wherein each segment has a shape possessing cylindrical symmetry, wherein the axis of symmetry of each individual segment points in a direction tangential to the rings peripheral shape (e.g. theta direction in a circular ring). For clarity, referring to the cylindrical symmetry of a segment means that the shape of a single segment possesses cylindrical symmetry around some axis of revolution; this is opposed to referring to the rotational symmetry of a ring which means that the multiple segments in an individual ring are arranged in a rotationally symmetric manner relative to each other. Such segments could be (but not limited to) in the shape of a cylinder (with its axis lying in the rings plane), a sphere, or an ellipsoid with two equal semi axes and one different semi-axis which is tangential to the ring's peripheral shape. In such a case one may rotate the segment around its own symmetry axis to tune the direction of magnetization of the segments in the r-z plane without changing the segments geometry.

The multiple magnet rings are arranged with reflectional asymmetry with respect to the longitudinal axis. In the context of the present disclosure and in the claims, the term “reflectional asymmetry with respect to the longitudinal axis” means that no plane perpendicular to the longitudinal axis is a plane of symmetry for the magnet array. In other words, the magnet array is not symmetric under flipping with respect to the longitudinal axis at any point along the axis. Reflectional asymmetry is also referred to as point asymmetry or mirror-image asymmetry. For brevity, any reference to “asymmetry” of the magnet array in the description below means the reflectional asymmetry defined above.

The multiple magnet rings are configured to jointly generate a magnetic field along a direction parallel to the longitudinal axis of at least a given level of uniformity inside the inner volume. The magnet array has each magnet ring generate a magnetic field having a rotational symmetry (continuous or discrete) with respect to an in-plane rotation of the ring around the longitudinal axis.

In some embodiments, each of the magnet rings of any of the disclosed magnet arrays has a shape comprising one of an ellipse, most commonly a circle, or of a polygon. The magnet rings are each made of either a single solid element or an assembly of discrete magnet segments. The magnet rings are pre-magnetized with a magnetization direction which is designed to maximize the uniformity of the magnetic field inside the inner volume and optionally minimize the safety zone defined by the area around the magnet for which the magnetic field exceeds 5 gauss.

In some embodiments, which are typically configured for head MRI applications, a disclosed asymmetric permanent magnet array can be described as comprising a first magnet assembly, comprising two or more magnet rings having a first inner diameter, and a second magnet assembly, comprising two or more magnet rings having a second inner diameter. The first inner diameter is larger than the maximal lateral diameter of the imaging volume and the second inner diameter is smaller than or equal to the maximal lateral diameter of the imaging volume.

Typically, the magnet rings lie in different longitudinal axis positions. The second magnet assembly is asymmetrically placed relative to the imaging volume. The asymmetric structure of the disclosed magnet array is thus optimized to fit a human head, in which physical access to an inner volume (which is the same as the imaging volume) containing the brain is through the first assembly but not the second. The first and second magnet assemblies are configured to jointly generate a magnetic field parallel to the longitudinal axis of at least a given level of uniformity inside the inner volume.

In some embodiments, the asymmetric magnet array is provided with at least two mixed-phase permanent magnet rings that are phase-dissimilar. In the context of the present invention, a mixed-phase magnet ring (MPMR) is defined as a magnet ring comprising multiple, repeating segments, each of which consisting of two or more phases, at least one of which is comprised of a permanent magnetic material.

A phase is defined as an element characterized by a particular combination of (i) material composition, (ii) geometric shape and relative position within the segment, and (iii) magnetization state. The magnetization state is represented by three components of magnetic moment, M=(Mr,Mθ,MZ), which are shared by corresponding phases in different segments, in the aforementioned cylindrical reference frame of coordinates. The materials of the various phases may be (but not limited to) permanent magnets, ferromagnetic, ferrimagnetic, paramagnetic, diamagnetic, antiferromagnetic or non-magnetic. The total magnetic field of an MPMR at any point is calculated by superposing the contributions of all phases in the ring which have nonzero values of M.

The phases fill the entire MPMR effective volume, which is defined as the volume of a polygonal annular ring of a minimum cross-sectional area, which just encloses all magnetic phases in the ring. The volumetric ratio of a phase is defined as the ratio of the phase volume to the effective volume of the MPMR.

Two MPMRs are said to be phase-similar if there is a one-to-one correspondence between the phases of the two rings for which (a) the volumetric ratios of corresponding phases are the same, (b) the magnetic permeabilities of corresponding non-permanent magnet phases are the same, and (c) the magnetization vectors of corresponding phases differ at most by a rotation through a constant angle in the r-Z plane common for all phases, and by a constant scaling factor in the magnetization magnitudes common for all phases. Thus, when two MPMRs are phase-dissimilar, the relative contribution of each individual phase in a given ring to the total magnetic field of that ring is different for the two rings. For example, with the aid of computerized magnetic field simulation tools, the phases of at least two MPMRs which are phase-dissimilar, and the magnetic moment directions of their permanent magnet phases, can be adjusted, or “tuned,” so as to optimize the uniformity of the total magnetic field inside an inner volume. These extra degrees of freedom are most advantageous when the array is subject to various geometric constraints (such as position of the rings, radial/axial thickness), which commonly arouse from mechanical or manufactural limitations. It will be appreciated that a solid magnet ring piece can be magnetized in an azimuthal repetitive manner so as to create repeating segments, with each segment magnetized with a different magnetization direction and/or strength. In the present context, such a magnet piece will be considered an MPMR where the phases share common material composition but differ in their magnetization states, even though mechanically there is no actual segmentation of the magnet ring. The same holds, for instance, for a solid magnet ring created with different material compositions where the composition changes in an azimuthal repetitive way. In such a case, different magnetic compositions area will be considered as different phases. The same holds for a solid magnet piece which has its axial thickness and/or radial thickness and/or cross section geometry vary azimuthally in a repetitive way. In this case the ring will be considered an MPMR with phases which differ by their geometry but share a common composition and magnetic state, even though there is no segmentation mechanically.

For a given weight of an asymmetric magnet ring array, using two or more phase-dissimilar MPMRs will result in a level of field uniformity inside the inner volume that is substantially higher than that achieved by the asymmetric array incorporating only one MPMR or several phase-similar MPMRs.

The various types of magnet rings and magnetic elements disclosed above are typically made of a strongly ferromagnetic material, such as an alloy of Neodymium, iron, and boron (NdFeB), whose Curie temperature is well above the maximum ambient operating temperature. Other material options include ferrites, samarium-cobalt (SmCo) magnets, or any other permanent magnet material. Depending on the design and type of ring, ring segments may have the shape of a sphere, a cylinder, an ellipsoid, or a polygonal prism with shapes such as a cuboid, a wedge, or an angular segment.

In some embodiments, a magnet array is provided that includes at least one magnet ring, which is rotationally symmetric and characterized by magnetization components M=(Mr,Mθ,MZ), having a finite component of magnetization along the azimuthal (θ) coordinate (i.e., a non-zero azimuthal projection of the magnetization) in addition to having a finite component (i.e., non-zero projection of the magnetization) of the magnetization in a longitudinal-radial plane. Such a magnet ring is named hereinafter “theta magnetic ring.” Including at least one such theta magnetic ring in the asymmetric array can improve uniformity inside the inner volume compared with that achieved by a magnet array of a same weight made solely of rotationally symmetric solid or segmented rings having magnetization solely in a longitudinal-radial plane.

In some embodiments the disclosed magnet array is used to utilize a mobile ambulance MRI. In some embodiments the ambulance is magnetically shielded with a high permeability material, as to provide a magnetically insulated cabin. In some embodiments the disclosed MRI device is combined with automatic algorithms to automatically detect stroke in a patient. In some embodiments the disclosed MRI device is used to perform MRI-guided brain thrombectomy preferably inside the ambulance.

The disclosed techniques to realize magnet arrays (e.g., using an asymmetric geometry, using two or more MPMR rings, using one or more theta rings, using cylindrically symmetric segments), separately or combined, enable the use of strong and uniform magnet arrays in applications that specifically require lightweight magnet solutions.

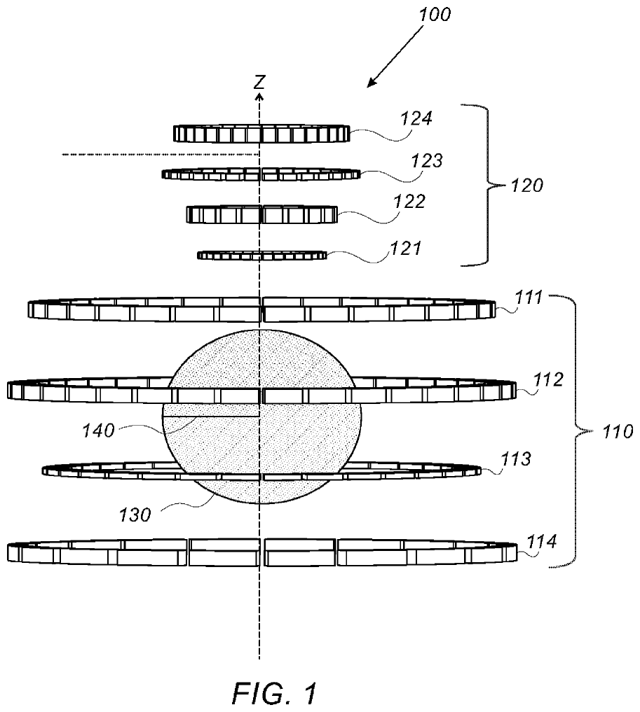

FIG. 1 is a perspective view of an exemplary asymmetric magnet array 100 comprising a first magnet assembly 110 and a second magnet assembly 120, according to an embodiment of the present invention. As seen, first and second magnet assemblies 110 and 120 each comprise permanent magnetic elements. In this case the magnetic elements form multiple magnet rings which are coaxial with a central longitudinal axis, denoted “Z-axis,” which passes through an inner volume 130. The multiplicity of magnet rings has variable transverse dimensions and variable displacements along the Z-axis. In FIG. 1, by way of example, first assembly 110 is shown as consisting of four magnet rings, 111-114, and second assembly 120 is shown as consisting of four magnet rings, 121-124. Each of the rings in assemblies 110 and 120 is either a solid ring or a segmented ring, i.e., a ring comprising discrete segments. The segments may have the shape of a sphere, a cylinder, an ellipsoid, or a polygonal prism, preferably cuboids. It will be appreciated that the rings may have any cross section including non-regular shape cross section. All segments belonging to a single ring share a common shape and material composition, as well as the same magnetic moment components in the longitudinal (Z), radial, and azimuthal directions. However, one or more of these characteristics may differ from one ring to another.

In case of a segmented ring, referring to the magnetic moment of a segment means that the segment is uniformly magnetized to a specific direction in space, its radial, longitudinal and azimuthal directions are calculated in the segment center of mass. In case of a solid ring, M varies continuously in space having azimuthal, radial, and longitudinal components independent of the azimuth coordinate. It will be appreciated that a solid magnet piece with a complex shape may be magnetized in a fashion that Mr,Mθ, or MZ changes as a function of Z, or R, in a gradual or stepped way, creating effectively several rings from a magnetization perspective, although mechanically composed of one continuous piece. In the present context, this sort of implementation is regarded as having multiple rings where their borders are determined by the magnetization perspective, rather than by mechanical segmentation. The peripheral shape of the rings may be any closed curve, such as a circle, ellipse, or polygon. In some cases, the choice of peripheral shape depends upon the cross-sectional shape of inner volume 130. It will be appreciated that a rotational symmetry of a ring, implies among others, that its peripheral shape is also rotationally symmetric (For example a shape of a circle, or an equiangular-equilateral polygon). In the special case where all rings are circular, the minimal inner radius of rings 111-114 of first assembly 110 (i.e. the smallest inner radius among the inner radiuses of rings 111-114) is denoted by R1, and the minimal inner radius of rings 121-124 of second assembly 120 (i.e. the smallest inner radius among the inner radiuses of rings 121-124) is denoted by R2. For a given target radius Ri, which, by way of example, has the lateral radius 140 of inner volume 130 that defines a maximal radius of a spheroid volume inside that is used for imaging and which the magnetic field has at least a given level of uniformity, the values of R1 and R2 satisfy the relationship Ri<R1, and 0≤R2≤Ri. In the case of R2=0, at least one of the rings of second assembly 120 is a solid disc. It is appreciated that assembly 120 may contain rings with inner radius larger than R2 and even larger than R1. The assemblies are separated in the Z direction with a gap which is typically (but not limited to) 0-10 cm. For the present purpose, if a ring extends in Z direction to both assemblies, one part of the ring will be considered as included in the first assembly while the other part in the second assembly. In this case the gap between arrays will be 0.

In an embodiment, in the asymmetric array, the minimal radius of the rings positioned on one side of the center of the inner volume is different from the minimal radius of the rings positioned on the other side of the center. The center of the inner volume can be defined in any suitable way, e.g., the center of the section of the longitudinal axis that lies within the inner volume. In addition, when the inner imaging volume is only partially enclosed by the array the center will be considered as the center of the section of the longitudinal axis that lies within the inner volume and inside the array. An array which obeys the former embodiment may be described as comprised of two sub-assemblies with different minimal inner radiuses as described above.

Inner volume 130 is a simply-connected region at least partially enclosed by assembly 110, which is typically an ellipsoid or a sphere. As shown, the inner volume 130 is enclosed by the magnet array 110, with rings 112-113 encircling inner volume 130. In an embodiment, inner volume 130 is an oblate ellipsoid with semi-axes approximately equal to 0.5 R1, 0.5 R1, and 0.3 R1. The parameters of such rings are not limited to the inner and outer radius of a ring, its Z displacement, or Z-axis thickness. In addition, magnetic moment angles are all optimized using a calculation method such as a finite element, finite difference, or analytical approach, combined with a gradient descent optimization algorithm to achieve the best uniformity, for a given field strength in the imaging volume, with a minimal weight. This is allowed due to the fact that each assembly contains a multiplicity of rings, all of which are optimized.

One aspect of the asymmetry of magnet array 100 is that different rings have different transverse dimensions and magnetic moment directions wherein the rings are arranged in an array having reflectional asymmetry with respect to the longitudinal axis (i.e., are asymmetrical with respect to Z-axis inversion). In the context of the present disclosure and in the claims, the term “reflectional asymmetry with respect to the longitudinal axis” means that no plane perpendicular to the longitudinal axis is a plane of symmetry for the magnet array. In other words, the magnet array is not symmetric under flipping with respect to the longitudinal axis at any point along the axis. Reflectional asymmetry is also referred to as point asymmetry or mirror-image asymmetry. For brevity, any reference to “asymmetry” of the magnet array in the description below means the reflectional asymmetry defined above. The asymmetry in the design is particularly advantageous when imaging inherently non-symmetrical specimens, such as the human head. For example, in one such case, it has been found that the rings belonging to assembly 110 may be primarily magnetized in a first given direction (e.g., the r-direction), whereas those belonging to assembly 120 may primarily magnetized in another direction (e.g., the z-direction).

Finally, the direction of magnetization of each individual ring may be optimized to obtain both uniformity in the inner volume as well as fringe field reduction so as to create a magnetic circuit which closes the field lines close to the magnet ring. In an embodiment, the discrete magnet segments are each pre-magnetized with a respective magnetization direction that minimizes a fringe field outside the magnet array.

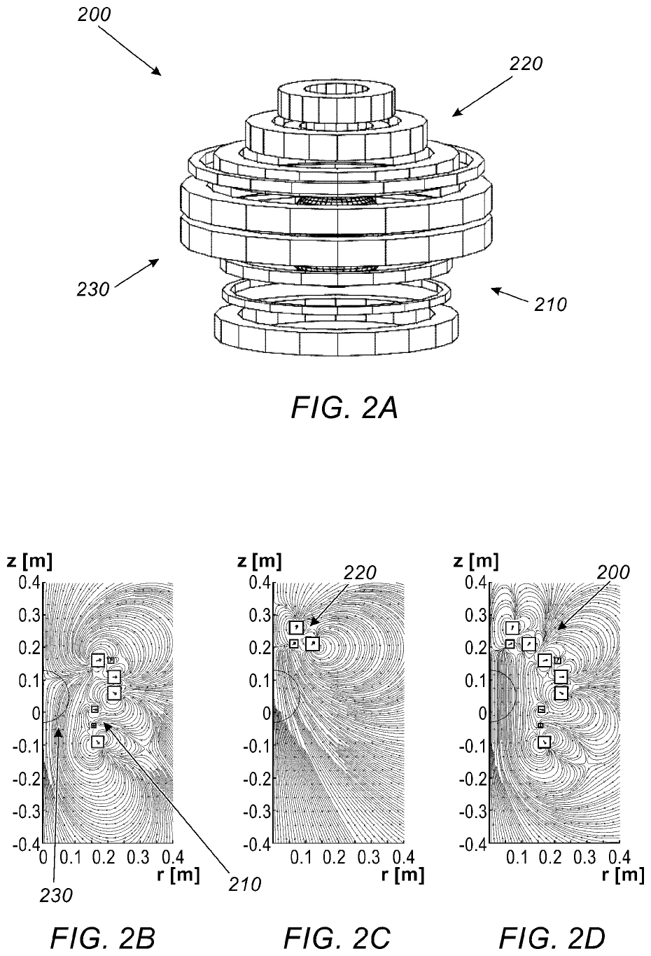

FIGS. 2A and 2B-2D are a perspective view of an asymmetric magnet array 200, and plots of magnetic field lines generated separately and jointly by the assemblies, respectively, according to another embodiment of the present invention. Uniformity is not evident by uniform density of the lines (as lines were drawn denser in the imaging zone for better details) rather by z-axis alignment of the lines.

As seen in FIG. 2A, an inner volume 230 is a simply-connected region at least partially enclosed by a first magnet assembly 210, which is typically an ellipsoid or a sphere. A second magnet assembly 220 of the asymmetric array, “caps” inner volume 230. As mentioned above, different rings may have different magnetization directions to optimize the uniformity and fringe field of the magnet array. For instance, one ring may have a magnetization vector in a direction substantially different (e.g., by more than 45 degrees) from another ring. For instance, the magnetization vectors of the permanent magnet segments may point primarily in the r direction in one ring, and primarily in the Z direction in another ring. Furthermore, two rings belonging to the same assembly may have substantially different magnetization directions. For instance, one ring of the first assembly may have its magnetization primarily in the r direction, another ring of the first assembly may have its magnetization in primarily the −z direction while a third ring of the first assembly may have its magnetization at −45 degrees in the r-z plane. In an embodiment, the two or more ring have a magnetization vector in a direction different by more than 45 degrees from one another.

In a particular case (not shown) it was found that the rings in assembly 210 are dispersed in their inner radius between 15 cm and 30 cm, and dispersed in their Z position in a length of 25 cm, while the rings in assembly 220 are dispersed in their inner radius between 0.05 cm and 30 cm, and dispersed in their Z position in a length of 12 cm, with the displacement between the two assemblies in the Z direction between 0 cm and 10 cm.

FIG. 2B shows the magnetic field lines of the field generated by first magnet assembly 210 (rings cross-sectionally illustrated by squares, each with a direction of magnetization of the ring in an r-z plane) inside and outside an inner volume 230. As seen, the field lines inside inner volume 230 are largely aligned along the z-axis, however they sharply bend at the top portion of volume 230, where the field becomes exceedingly non-uniform.

FIG. 2C shows the magnetic field lines of the field generated by second magnet assembly 220 inside and outside an inner volume 230. As also seen here, the field lines inside inner volume 230 are largely aligned along the z-axis. However, they tilt opposite to the field lines of FIG. 2B with respect to the z-axis, and become exceedingly non-uniform at a bottom portion of volume 230.

As seen on FIG. 2D, when combined into a full array 200, assemblies 210 and 220 compensate for each other's field non-uniformity, to achieve a uniform magnetic field along the z-axis to a better degree than a prespecified threshold.

FIGS. 2A-2D show an exemplary array containing ten rings. It will be appreciated that the array may contain more rings (e.g., several tens or hundreds of rings) which are all optimized as described above. The more rings contained in the array, the better magnet performance can be achieved (e.g., higher uniformity level, larger magnetic field or larger imaging volume). The improved performance comes with the drawback of increased complexity and production cost of the array due to the large number of elements. Thus, a practitioner skilled in the art should consider the required number of rings according to the specific application.

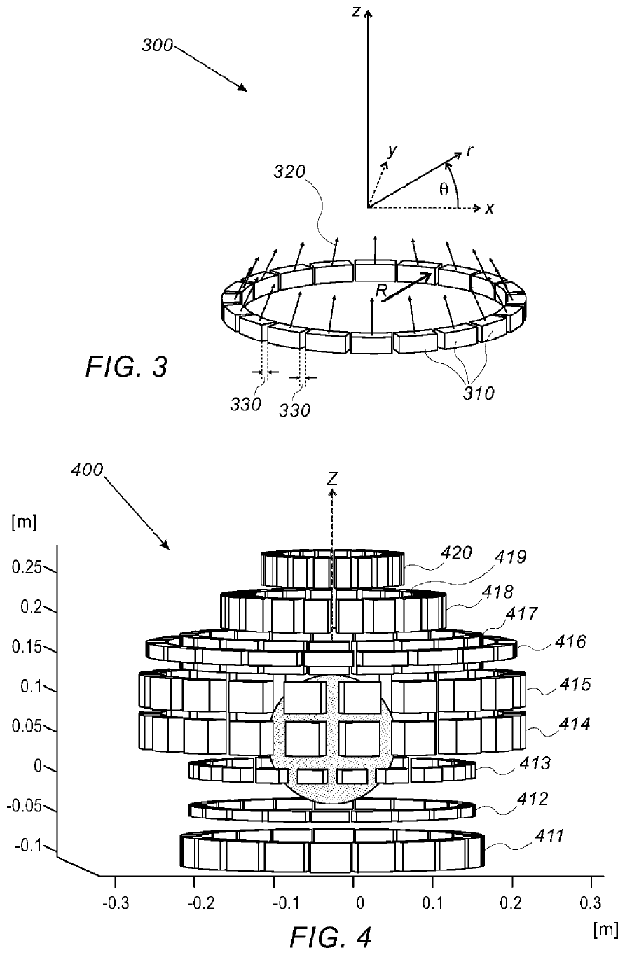

FIG. 3 is a perspective view of a single segmented magnet ring 300, which may be any one of the rings in magnet arrays 100 and 200 of FIGS. 1 and 2, according to an embodiment of the present invention. In FIG. 3, each magnet segment 310 has a magnetization vector 320 lying in the r-Z plane, with similar longitudinal (Z) and radial (r) components. Furthermore, each segmented ring possesses rotational symmetry with an azimuthal period equal to 360/N degrees where N is the number of segments in the ring. (For a solid ring, i.e., for N→∞, the rotational symmetry is continuous). In some embodiments, the disclosed rings have rotational symmetry of an order N≥8. It will be appreciated that the disclosed array contains rings with rotational symmetry and hence the result magnetic field is along the longitudinal axis. It is possible however to incorporate in the asymmetric array rings which are non-rotationally symmetric in a fashion that optimizes the fringe field and uniformity in the inner volume. In such a case the magnetic field may be along an arbitrary axis. Although such an array may be substantially worse than a rotationally symmetric array, the use of asymmetry with rings as disclosed may substantially improve uniformity of the array compared to a symmetric one.

Discrete segments 310 are equally spaced and attached to one another using, for example, an adhesive, which is preferably non-electrically conducting, or are held together mechanically with gaps 330 between adjacent segments filled by (but not limited to) a preferably insulating material. It will be appreciated that the rotational symmetric segmented rings may also include a combination of more than one type of segments. For thermal stability of all of ring 300, it is preferable that the adhesive or gaps consist of a material which is also thermally conductive, such as silicon oxide, silicon nitride, or aluminum oxide. Individual magnet segments 310 may be made of the aforementioned strongly ferromagnetic materials, whose Curie temperature is well above the operating temperature of an associated system that includes such elements as an array 200, e.g., a mobile MRI system.

It will be appreciated that the descriptions in FIGS. 1-3 are intended only to serve as examples, and that many other embodiments are possible within the scope of the present invention. For example, rotation of the magnet moment vector in the r-z-O plane can be achieved, in an alternative embodiment, by rotating the individual magnet segments 310 through a distinct angle of rotation, which may be different for different rings. Further, magnet arrays 100 and 200 may be combined with either a static or dynamic shimming system, to further improve field uniformity inside inner volumes 130 and 230, respectively. When dynamic shimming or gradient pulse fields are used, the presence of electrically insulating adhesive or empty gaps between adjacent magnet segments 310 helps to minimize the negative effects of eddy currents on field uniformity. Furthermore, magnet arrays 100 and 200 may be combined with resistive coils placed concentric to the z-axis, in order to enhance the magnetic field strength inside inner volumes 130 and 230.

### Magnet Array Including Mixed Phase Magnet Rings

FIG. 4 is a perspective drawing of a magnet array 400 of phase-dissimilar mixed phase magnet rings (MPMRs), according to an embodiment of the present invention. As seen, by way of example, array 400 comprises ten magnet rings 411-420 which are coaxial with a central Z-axis passing through an inner volume 430. The different rings are located at different positions along the Z-axis and, in general, have different transverse dimensions, radial thicknesses, and axial thicknesses. As seen, magnet array 400 has reflectional asymmetry with respect to the longitudinal axis (i.e., is asymmetric with respect to Z-axis inversion). In the context of the present disclosure and in the claims, the term “reflectional asymmetry with respect to the longitudinal axis” means that no plane perpendicular to the longitudinal axis is a plane of symmetry for the magnet array. In other words, the magnet array is not symmetric under flipping with respect to the longitudinal axis at any point along the axis. Reflectional asymmetry is also referred to as point asymmetry or mirror-image asymmetry. For brevity, any reference to “asymmetry” of the magnet array in the description below means the reflectional asymmetry defined above.

In addition, inner volume 430 may be interior (as shown) or at least partially extending exterior in the z-direction (not shown) to magnet array 400. Furthermore, the disclosed magnet array may or may not be combined with a yoke.

Ring 411 exemplifies an MPMR having cuboid-shaped permanent magnet elements (i.e. phase 1) separated by relatively small non-magnetic gaps (i.e. phase 2). Ring 413 exemplifies an MPMR having cuboid shaped permanent magnet elements (i.e. phase 1) separated by relatively large non-magnetic gaps (i.e. phase 2). Clearly, the fraction of the total ring volume occupied by non-magnetic gaps is small in the case of ring 411 and relatively large in the case of ring 413. Thus, rings 411 and 413 are MPMRs that are phase-dissimilar and array 400 may contain many phase-dissimilar MPMRs.

Furthermore, ring 411 may also have a magnetization vector in a direction substantially different (e.g., by more than 45 degrees) from ring 413. For instance, the magnetization vectors of the permanent magnet segments may point in the −Z direction in ring 411, and −45 degrees in the r-Z plane in ring 413. In an embodiment, the two or more mixed-phase magnet rings contain only one magnetic phase with a magnetization vector in a direction different by more than 45 degrees from one another. Each MPMR ring possesses rotational symmetry with an azimuthal period equal to 360/N degrees where N is the number of segments in the ring. (For a continuous ring, i.e., for N→∞, the rotational symmetry is continuous). In some embodiments, the disclosed MPMR rings have discrete rotational symmetry of an order N≥8.

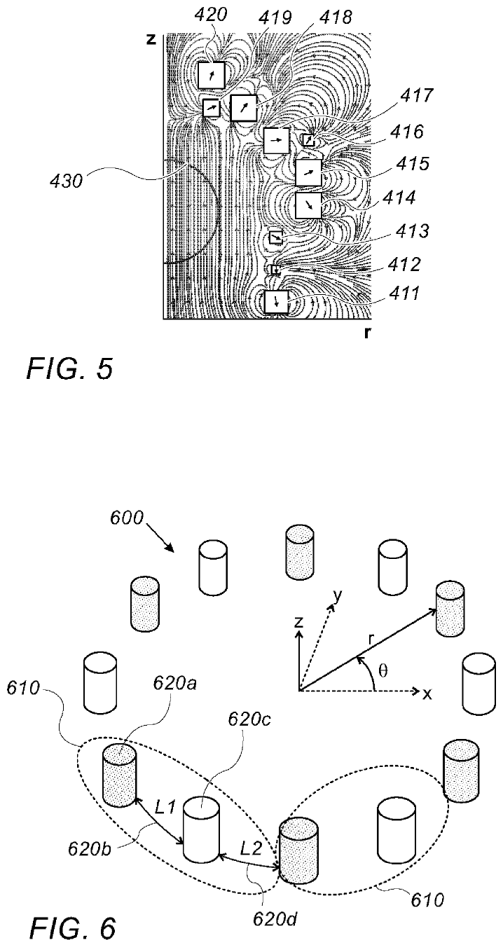

FIG. 5 is a plot of magnetic field lines generated by magnet array 400 of FIG. 4, according to an embodiment of the present invention. Uniformity is not evident by uniform density of the lines (as lines were drawn denser in the imaging zone for better details) rather by z-axis alignment of the lines. MPMR array 400 can achieve, at a same array weight, a more uniform magnetic field along the z-axis, compared with, for example, that achieved with arrays 100 and 200. Moreover, the uniform field extends radially almost to the rings. Such lightweight MPMR arrays may be therefore particularly useful for mobile MRI applications, such as an MRI ambulance.

FIGS. 4 and 5 show an exemplary array 400 containing ten MPMRs. It will be appreciated that the array may contain more MPMRs (e.g., several tens or hundreds of MPMRs) which are all optimized as described above with many of them phase dissimilar. The more rings contained in the array, the better magnet performance can be achieved (e.g., higher uniformity level, larger magnetic field or larger imaging volume). The improved performance comes with the drawback of increased complexity and production cost of the array due to the large number of elements. Thus, a practitioner skilled in the art should consider the required number of MPMRs according to the specific application. It is appreciated that an array may contain lots of rings e.g. 3, 4, 5, 6, 7, 8, 9, 10, which are mutually phase dissimilar MPMRs.

FIG. 6 is a perspective drawing of an exemplary MPMR 600 according to an embodiment of the invention. The ring consists of six repeating segments 610, each of which has four elements: 620a, 620b, 620c, and 620d. In an embodiment, elements 620a are made of the aforementioned strongly magnetic materials. Elements 620a are typically pre-magnetized with specific values for the components of magnetic moment. The shape of element 620a may be cylindrical, as shown in FIG. 3, or some other shape such as a sphere, an ellipsoid, a cuboid, or a polygonal prism.

Element 620c typically has a different phase from element 620a. For example, it may have the same material composition and geometric shape as element 620a, but differ in one or more components of the magnetic moment, M. Alternatively, element 620c may consist of a non-ferromagnetic material, such as a ferrimagnetic, paramagnetic, or non-magnetic material, in which case the phase of element 620c differs from that of element 620a, by virtue of its different material composition. Element 620b fills a gap of length L1 separating element 620a from element 620c; similarly, element 620d fills a gap of length L2 separating element 620c from element 620a of the adjacent segment, as shown in FIG. 6. Often, for thermal stability of MPMR 600, it is preferable that elements 620b and 620d consist of non-magnetic, electrically non-conducting materials which are at least moderately thermally conductive, such as silicon oxide, silicon nitride, or aluminum oxide.

In order to further illustrate the concept of phase-similar MPMR's, consider an MPMR 600 in which elements 620a and 620c have axial magnetizations M0 and −M0, respectively. Next, consider a different MPMR 600* (not shown) which is the same as MPMR 600 in all respects, except that elements 620a* and 620c* have radial magnetizations 2M0 and −2M0, respectively. Since MPMR 600 can be transformed into MPMR 600* by a common rotation of the magnetic moment by 90° in the r-Z plane followed by multiplication by a common scale factor of two, the two MPMR's are considered to be phase-similar. For each ring one may define the effective strength of the ring by the magnitude of the volume averaged r-Z projection of magnetization vector divided by the largest magnetization magnitude of all permanent magnet phases. The parameter has a value between 0 and 1; and has the qualitative meaning of how effective a ring produces a magnetic field nearby. When two rings are not phase similar, they may have different relative effective strengths and different contributions to the magnetic field.

Generally, adjacent elements in an MPMR are held together by mechanical means or by adhesives. If the total volume occupied by adhesive layers is small, e.g., less than 1% of the total volume of the ring, then the adhesive layers need not be treated as an additional phase for the purpose of magnetic field calculations. Small adjustments in the segments positions and angles may be carried out to compensate for the segments' imperfections and residual inhomogeneity.

It will be appreciated that the above descriptions are intended only to serve as examples, and that many other embodiments are possible within the scope of the present invention. For example, magnet array 400 may be combined with either a static or dynamic shimming system to further improve field uniformity inside inner volume 430. When dynamic shimming or gradient pulse fields are used, the presence of electrically insulating material in the gaps between adjacent magnet elements helps to minimize the deleterious effects of eddy currents on field uniformity. Furthermore, magnet array 400 may be combined with resistive coils placed concentric to the z-axis, in order to enhance the magnetic field strength inside inner volume 430.

In the example embodiments described herein, the mixed-phase rings are part of an asymmetric magnet array. In alternative embodiments, however, mixed-phase rings may be used also in symmetric arrays or any other type of magnet array, with or without a yoke to enhance their uniformity. In addition, the example magnet array described herein contains multiple rings coaxial with a common axis. It is however possible to combine the described array with one or more additional ring arrays for which the rings are coaxial with one or more different axes which are at an angle from the first longitudinal common axis. The combination of arrays jointly create a magnetic field in an arbitrary direction in space. The additional ring arrays may also contain mixed phase rings, those rings however are defined according to their own cylindrical coordinate system with a z′ axis defined as their own common coaxiality axis. It is possible, for example, to have two arrays of rings with respective coaxiality axes that differ by 45 degrees from one another. Each array may contain two or more phase-dissimilar MPMRs and may be optimized to obtain a field substantially uniform in the inner volume along each of the array axis. The combination of the two arrays results in a homogeneous magnetic field in a direction which is between the first and second longitudinal axes.

Although the embodiments described herein mainly address mobile MRI application, the methods and systems described herein can also be used in other applications, such as aerospace applications, that require strong, uniform and lightweight magnets such as scanning electron microscopes (SEM).

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

### Magnet Array Including Theta Magnet Rings

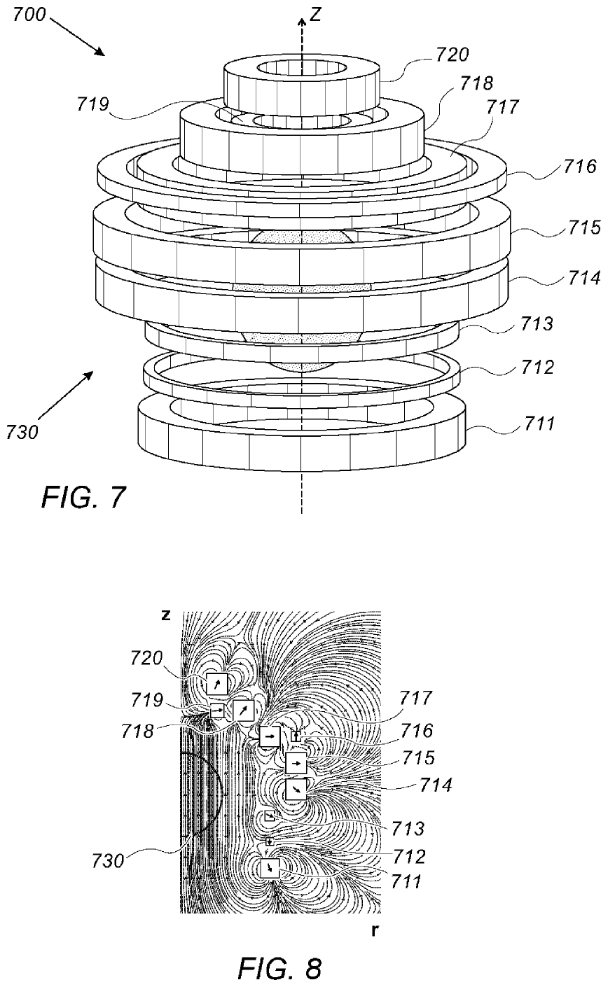

FIG. 7 is a perspective drawing of an asymmetric magnet array 700 comprising three theta magnetic rings (712, 713, 719), according to an embodiment of the present invention. By way of example, magnet array 700 comprises ten magnetic rings 711-720 surrounding an axis Z which passes through an inner volume 730. Some of the magnetic rings may be solid, and some may be segmented and optionally have gaps between adjacent magnetic segments. The rings are located at different positions along the Z-axis and, in general, have different transverse dimensions, radial thicknesses, and axial thicknesses.

As in the above shown arrays, magnet array 700 defines a standard cylindrical coordinate system. Each magnetic ring has a magnetization which is rotationally symmetric and characterized by magnetization components M=(Mr, Mθ, MZ). All the segments within a given segmented ring have the same magnetization components represented by three components of magnetic moment, M=(Mr, Mθ, MZ), in the aforementioned cylindrical reference frame of coordinates. Consequently, each segmented ring possesses rotational symmetry with an azimuthal period equal to 360/N degrees where N is the number of segments in the ring. In case of a segmented ring, referring to the magnetic moment of a segment means that the segment is uniformly magnetized to a specific direction in space, and its radial, longitudinal and azimuthal directions are calculated in the segment center of mass. In case of a solid ring, M varies continuously in space and has azimuthal, radial, and longitudinal components independent of theta. The magnetization M is generally different for different rings. At least one of the magnetic rings in the magnet array is a “theta magnetic ring;” that is, it has a non-zero projection of the magnetization in the theta direction (Mθ≠0) in addition to a non-zero projection of the magnetization in the r-Z plane. The non-zero projection on the r-Z plane is essential as a magnet ring with only azimuthal magnetization does not produce a substantial magnetic field. Essentially, the introduction of a non-zero theta component in a given ring has the effect of reducing the relative contribution of that ring to the total magnetic field inside the imaging volume, thus providing extra degrees of freedom which are unrelated to the geometry of the rings. These extra degrees of freedom are most advantageous when the array is subject to various geometric constraints (such as position of the rings, radial/axial thickness), which commonly arouse from mechanical or manufactural limitations. With the aid of computerized magnetic field simulation tools, a designer can adjust, or “tune,” the magnitude of the non-zero theta component in the theta magnetic ring(s), together with geometric properties of all the magnetic rings (such as height, outer radius, inner radius, thickness, and z-axis position) so as to achieve a high level of magnetic field uniformity, or a large inner volume, as required, for example for portable head MRI systems.

For example, rings 712, 713, and 719 may be theta rings having magnetization directions in cylindrical coordinates (Mr, Mθ, MZ) given by (0, √3/2,−1/2), (1/√3,1/√3,−1/√3), and (1/√2, 1/√2, 0) respectively. In an embodiment, the one or more magnet rings with the finite component of magnetization along the azimuthal (θ) coordinate and the rest of the rings, are configured to jointly generate the magnetic field with at least a given level of uniformity inside the inner volume.

The magnetic segments of magnetic rings 711-715 can be made of the aforementioned strongly magnetic materials. The segments typically are pre-magnetized with specific values for the components of magnetic moment. The shape of the segments may be any of the aforementioned segment shapes (e.g., wedge or angular segment).

The disclosed introduction of a non-zero theta component in the magnetization vector (Mθ≠0) of at least one ring in an array of magnetic rings can greatly enhance the uniformity of the magnetic field inside the inner volume of the array, or alternatively, greatly enlarge the inner volume for a given level of uniformity. This advantage applies to solid rings which comprise a solid magnet piece with spatially continuous magnetization. It also applies to segmented magnetic rings with segments which are contiguous with no gaps, as well as to rings whose segments are separated by air gaps or gaps filled with a non-magnetic material. It is appreciated that the gaps may be also filled with materials which are not permanent magnets but has some non-trivial magnetic permeability such as (but not limited to) paramagnets, antiferromagnets, diamagnets, ferromagnets, and ferrimagnets.

FIG. 8 is a plot of magnetic field lines generated by magnet array 700 of FIG. 7, according to an embodiment of the present invention. Uniformity is not evident by uniform density of the lines (as lines were drawn denser in the imaging zone for better details) rather by z-axis alignment of the lines. As seen, array 700 achieves a uniform magnetic field along the z-axis with z axis alignment which extends almost to the rings. While not visible, the theta rings improve uniformity. Therefore, including a few theta magnet rings in an asymmetric array of ring magnets may therefore be particularly useful for mobile MRI applications, such as an MRI ambulance.

FIGS. 7 and 8 show an exemplary array containing a total of ten rings, from which three are theta rings. It will be appreciated that the array may contain more theta rings (e.g., several tens or hundreds of rings) which are all optimized as described above. The more theta rings contained in the array, the better magnet performance can be achieved (e.g., higher uniformity level, larger magnetic field or larger imaging volume). The improved performance comes with the drawback of increased complexity and production cost of the array due to the large number of elements. Thus, a practitioner skilled in the art should consider the required number of rings according to the specific application.

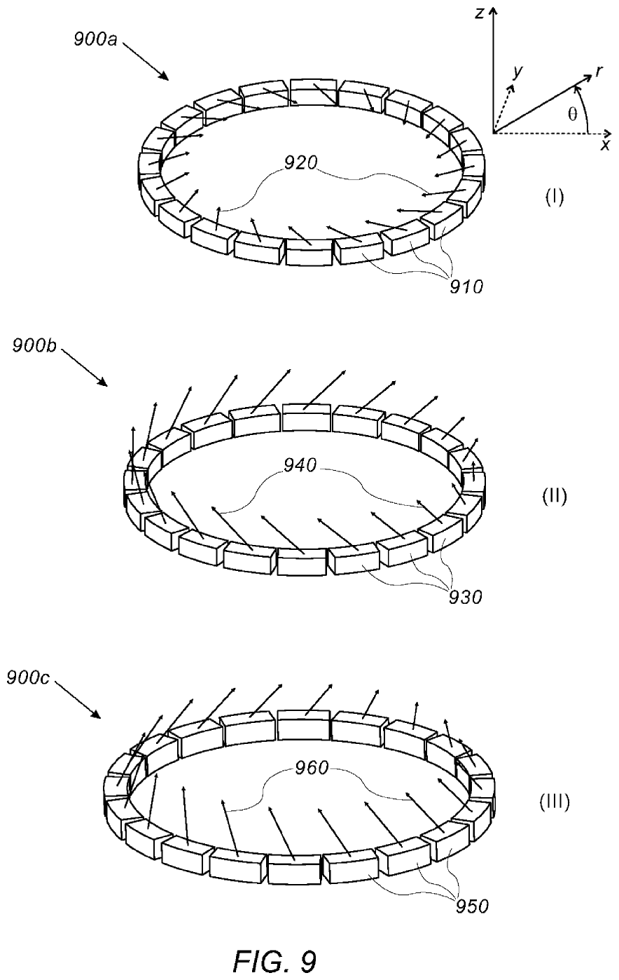

FIG. 9 is a perspective view of theta magnet rings, which may be any one of the rings in magnet array 700 of FIG. 7, according to embodiments of the present invention. FIG. 9 (I) shows a perspective drawing of a first exemplary theta magnetic ring 900a. In FIG. 9 (I), the theta magnetic ring comprises twenty cuboid magnetic segments 910. The magnetic moment of each segment 920 has a zero axial (Z) component and non-zero radial (r) and theta (θ) components, as illustrated by arrows 920. FIG. 9 (II) shows a perspective drawing of a second exemplary theta magnetic ring 900b, comprising twenty cuboid magnetic segments 930. The magnetic moment of each segment 930 has a zero radial (r) component and non-zero axial (Z) and theta (θ) components, as illustrated by arrows 940. FIG. 9 (III) shows a perspective drawing of a third exemplary theta magnetic ring 900c, comprising twenty cuboid magnetic segments 950. The magnetic moment of each segment 950 has non-zero radial (r), theta (θ) and axial (Z) components, as illustrated by arrows 960.

It will be appreciated that the above descriptions are intended only to serve as examples, and that many other embodiments are possible within the scope of the present invention. For example, magnet array 700 may be combined with either a static or dynamic shimming system, to further improve field uniformity inside imaging volume 730. In addition, the presented magnet array is asymmetric however, the theta rings may be used also in symmetric arrays or any other type of magnetic array, with or without a yoke to enhance their uniformity.

In addition, it is possible to combine the described array (having multiple rings coaxial with a common axis) with one or more additional ring arrays for which the rings are coaxial with one or more different axes which are at an angle from the first longitudinal common axis. The combination of arrays jointly creates a magnetic field in an arbitrary direction in space. The additional ring arrays may also contain theta phase rings, those rings however are defined according to their own cylindrical coordinate system with a z′ axis defined as their own common coaxiality axis. It is possible, for example, to have two arrays of rings with coaxiality axes that differ by 45 degrees from one another. Each array may contain one or more theta rings and may be optimized to obtain a field substantially uniform in the inner volume along each of the array axes. The combination of the two arrays results in a homogeneous magnetic field in a direction which is between the first and second longitudinal axes.

It is appreciated that in all aforementioned arrays, the angle of magnetic moment of the rings relative to the longitudinal axis may be a non-monotonic function of the ring's axial position or radial position as shown in FIGS. 2A-2D, FIG. 5 and FIG. 8. Furthermore, the magnetic moment direction is not constrained to follow any predetermined law. For example, a law wherein the magnetic moment angle relative to the longitudinal axis is twice the angle between the longitudinal axis and the line connecting the center of the imaging volume and the ring, is not satisfied. The arrays shown in FIGS. 2,5,7 clearly deviate from such a law. Furthermore, the magnet array in FIGS. 1,2,5,7 clearly deviates from a spherical shape.

### Magnet Array Including Rings Made of Segments with Cylindrically Symmetric Shape

All aforementioned rings which are rotationally symmetric, (MPMRS, theta rings and rotationally symmetric segmented rings) are preferably made of segments which have cylindrically symmetric shapes, i.e., each individual segment possesses a cylindrical symmetry around its own axis of symmetry. The axis of symmetry of each segment lies in the ring's plane, tangential to the peripheral shape of the ring e.g., in the azimuthal (theta) direction for a circular ring. By rotating each segment around its own axis of symmetry one may adjust the magnetization direction of the segments in the r-z plane without changing the geometry of the magnetic ring. The ring as a whole has rotational symmetry with respect to the longitudinal axis, thus, corresponding segments in a ring are adjusted to have the same magnetic moment direction in the r-z plane, i.e. to have the same magnetic moment radial component Mr, axial component Mz, and tangential component Mθ. A special case is a sphere that could be adjusted in three axes (θ, r, z) and thus also the theta component could be adjusted without changing the ring's geometry. This is especially useful to adjust theta rings. The segments are homogenously magnetized, in a general direction. In an embodiment of the present invention, the segments are magnetized in a direction perpendicular to the symmetry axis of each segment. In such a case the magnetization of the ring composed of such segment will lie in the r-z plane. Alternatively, a segment may have a component of magnetic moment in a direction parallel to his axis of symmetry. In such a case the ring composed of such segments will have a magnetization vector which has a component in the r-z plane as well as a component along azimuthal (θ) direction.

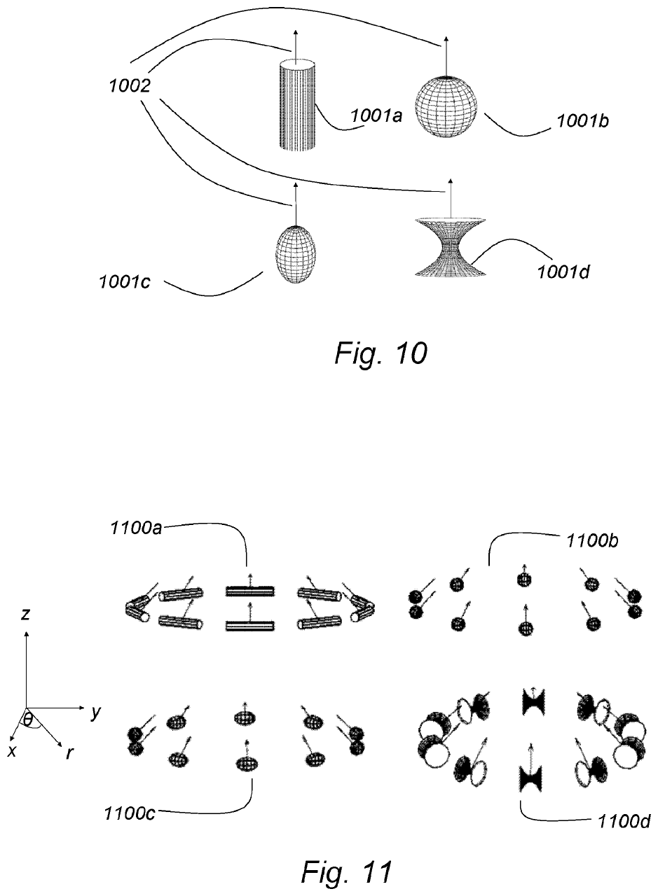

FIG. 10 shows a perspective view of exemplary permanent magnet shapes possessing cylindrical symmetry. The axis of symmetry for each segment is shown by arrows 1002 and is denoted herein by S. FIG. 10 shows a cylinder (1001 (a)), a sphere (1001 (b)), an ellipsoid (1001 (c)) with two equal semi axes and one different semi-axes which is in the direction of axis of symmetry S, and a general shape having cylindrical symmetry around axis S (1001 (d)).

FIG. 11 shows perspective views of exemplary magnet rings possessing rotational symmetry around the longitudinal (z) axis; 1100 (a), 1100 (b), 1100 (c), 1100 (d), which are composed of the segments shown in FIG. 10. Ring 1100(a) is composed of segments similar to segment 1001(a), ring 1100(b) is composed of segments similar to segment 1001(b), ring 1100(c) is composed of segments similar to segment 1001 (c), and ring 1100(d) is composed of segments similar to segment 1001(d). The arrows in FIG. 11 denote the magnetization direction of the segments; in this case, the magnetization radial, axial, and azimuthal components are the same in all segments and point to a general direction in the r-z-theta plane (i.e. not necessarily to the r direction, or z direction) e.g. in a 45 degrees angle in the r-z plane. As shown, the axis of symmetry (S) of each segment is tangential to the peripheral shape of the ring such that rotation of each segment around his own axis of symmetry results in rotation of the magnetization direction in the r-z plane. This provides the technical advantage of tuning the magnetization of the ring without changing the physical geometry of the ring.

It is appreciated that although the examples herein show a ring with only one type of segments, a ring may contain two or more types of permanent magnet segments (e.g., in an MPMR) which have different shapes. Preferably, all segments' shapes have cylindrical symmetry, with the axis of symmetry of each individual segment lying tangent to the peripheral shape of the ring, as described above.

The case discussed above, is the case where the symmetry axis S of each individual segment lies tangential to the peripheral shape of the ring. This is the preferred case. For circular rings and when the segments are equally distributed identical segments, the direction of the S axis is the theta direction. However, it is possible for the direction of S, to be in a general direction which is not tangent to the peripheral shape of the ring, as long as the ring as a whole still fulfills the rotational symmetry condition around the longitudinal axis; i.e., in the cylindrical coordinate system defined for a ring, the S axis has azimuthal (theta), radial (r), and axial (z) components which are common to all corresponding segments. For clarity, the axial, azimuthal, and radial directions are calculated at the segment's center of mass. For instance, the segments may lie obliquely such that the S axis of each segment has a constant angle from the ring's lateral plane. For example, in a circular ring, the S axis may have a radial and azimuthal component, or axial and azimuthal components. It may also have, radial, azimuthal and axial components all together. When the S axis has only radial, or axial components, the magnetic moment of the segment can be tuned in the z-theta, and r-theta planes, respectively, by rotating each segment around his own S axis. When the S axis is in a general direction, the magnetic moment of the segments can be rotated around this general S axis. If the ring is an MPMR and is composed of several magnetic phases each with segments shapes which possess cylindrical symmetry, each phase may have its S axis in a different direction under the condition that rotational symmetry of the ring still remains, i.e., the S axis of corresponding segments of the same phase has a radial, azimuthal and axial components common to all segments belonging to the same phase. Note that this condition is essential, for a ring to be an MPMR, as segments corresponding to the same phase share the same geometry.

It will be appreciated that the aforementioned segments can be used also in non rotationally-symmetric rings. It is also appreciated that it is possible for only some of the segments of a given ring to possess cylindrical symmetric shape wherein the symmetry axis of each of them lying in a direction with a component tangential to the peripheral shape of the ring. In an extreme case, a ring may contain only one such segment. In addition, a ring as disclosed may be combined in any type of magnet array.

### Mobile Ambulance Brain MRI

The aforementioned magnet may be used to utilize mobile ambulance brain MRI, wherein the human head slides through the bottom opening of the magnet arrays shown in FIGS. 1,2,4,7 and the brain is substantially contained in the imaging volume, and preferably has the same lateral, and axial size as the imaging volume. As the head is fully enclosed by the magnet with some of the rings encircling the head, it is preferable to have holes between the rings, and an axial hole in the upper part of the magnet (as shown in FIGS. 1,2,4) for ventilation and better patient experience (e.g. R2 defined of the second array is larger than zero).

The aforementioned magnet may be combined with a suitable gradient field system and an RF MRI coil, to obtain an MRI system capable of head imaging in various protocols (e.g. T1, T2, diffusion weighted, MR spectroscopy etc). The small size of the magnet allows it to be placed in an ambulance. This technical advantage is especially important in life threatening situations such as brain hematoma, or stroke. It is thus preferable to use the MRI system in a diffusion weighted protocol in order to diagnose an ischemic brain stroke as soon as possible while the patient is in the ambulance.

The system may be combined with an automatic algorithm which analyzes the acquired data and provides an automatic diagnosis, e.g., whether the imaged patient is experiencing a stroke. The algorithm may also extract various parameters such as the stroke location, the size of the penumbra, the size of damaged area, chance of large vessel occlusion (LVO) etc. The automatic algorithm may use (but not limited to) artificial intelligence, machine learning algorithms, convolutional neural networks (CNNs), classical image processing algorithms, supervised, unsupervised and reinforcement learning algorithm etc. Such an algorithm can obtain additional inputs such as (but not limited to) a stroke severity score determined by the medical personnel (e.g. the NUBS score), the onset of symptoms (if known), whether the stroke is a wake-up stroke, age of the patient etc. Taking into account such inputs may lead to a higher degree of sensitivity or specificity. The algorithm preferably obtains relevant medical data as input such as prior surgeries, prior strokes, anticoagulants medications taken by the patient, hemophilic disease history, high blood pressure history. The algorithm then automatically assesses based on all input data the stroke subtype and patient eligibility to various treatments such as recombinant tissue plasminogen activator (rTPA) or brain thrombectomy. Furthermore, the algorithm preferably includes a probabilistic model which takes into account data about optional hospital or stroke centers, including (but not limited to) their distance from the ambulance location, estimated time of arrival of the ambulance to each center, available treatments in each center, crowdedness of the stroke unit and availability of treatments (such data may be updated directly from an automatic system of the hospital in real time), in order to assess based on all available data the center/hospital which is best likely to provide the quickest and best treatment suitable for the medical condition of the patient. Such a system will have the benefit of saving secondary transfers when the patient is first transferred to a hospital and then transferred again to another hospital which provides the relevant treatment.

When a patient is diagnosed with a stroke it is possible to treat him inside the ambulance. Such treatment includes for example, injecting him recombinant tissue plasminogen activator (rTPA), or alternatively performing a brain thrombectomy. Such brain thrombectomy may be performed while the patient is imaged in the device in an MRI guided manner. The MRI system may also be used to navigate an MRI compatible catheter through the body arteries using gradient system and magnetic field sensing on the catheter to locate its position. Access to the patient may be provided through holes between the magnet rings, through the bottom or upper axial holes. Life support measures may also be provided to the patient such as oxygen through aforementioned holes. A camera to monitor the patient condition while being inside the magnet is also preferable.

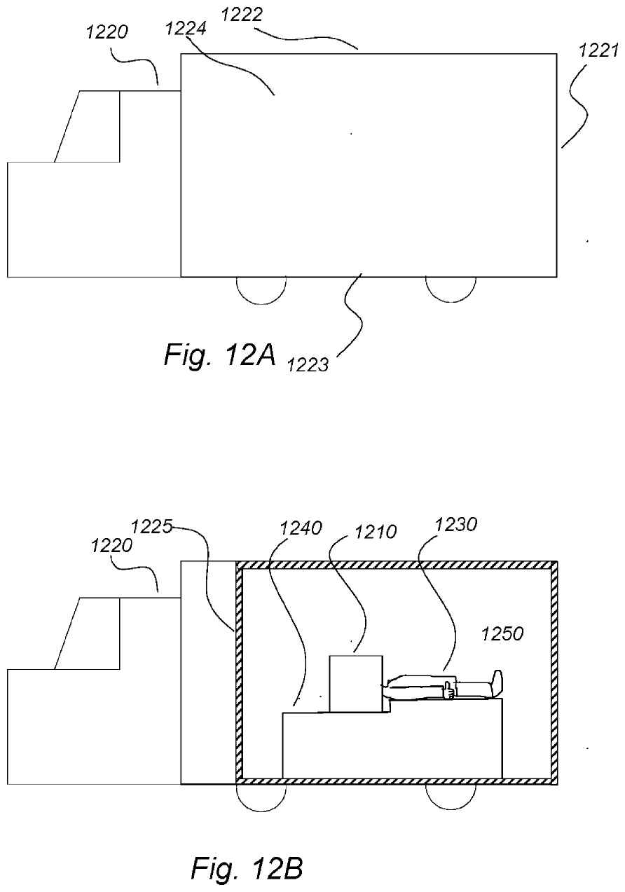

FIG. 12B shows a schematic cross-sectional side view of an ambulance according to an embodiment of the invention. As shown, an MRI device 1210 is placed inside an ambulance 1220. The patient, denoted as 1230, has its head inserted to the device, preferably while the patient is lying on an MRI compatible bed 1240. A magnetic shielding on the ambulance is preferable. Passive magnetic shielding is composed of layers (at least one layer) of high magnetic permeability material with optionally non-magnetic spacers between the layers to prevent external magnetic field to penetrate to the ambulance and also prevent leakage of magnetic field from interior of the ambulance to the external environment. In the present context, high permeability coating refers to a coating made of a material or several materials having a high magnetic permeability, such as (but not limited to) steel, mu-metal, permalloy (alloys of Fe and Ni) etc. Such magnetic shielding is attached to the exterior or interior part of the ambulance, creating a cabin (denoted by 1250) which is magnetically insulated from the external environment. The shape of the created cabin is preferably cubic but could be any other shape. The cabin is surrounded entirely by high permeability material, and as a result, provides an inner volume which is magnetically insulated from external environment.

FIG. 12A shows a schematic side view of the ambulance according to an embodiment of the invention. The rear door of the ambulance is denoted as 1221, the ceiling of the ambulance is denoted as 1222, the floor of the ambulance is denoted as 1223 and the front and back side walls of the ambulance are denoted as 1224. Note that FIG. 12A only shows the front side wall of the ambulance (1224) but not the back because of the drawing view. In order to prevent magnetic field penetration, the rear door of the ambulance (1221), at least part of the ceiling of the ambulance (1222), at least part of the floor of the ambulance (1223), and the at least part of the walls of the ambulance which creates the walls of the cabin, is preferably coated with the high permeability material shown in FIG. 12B as a dashed pattern. In addition, an interior barrier coated with high permeability material between the magnetically insulated cabin and other parts of the ambulance may be placed (denoted by element 1225 in FIG. 12B). In addition, the high permeability coating on front and back wall of the ambulance is not shown in FIG. 12B, but as mentioned above, such coating should preferably be present on at least the part of the walls of the ambulance which creates the walls of the magnetically insulated cabin 1250.

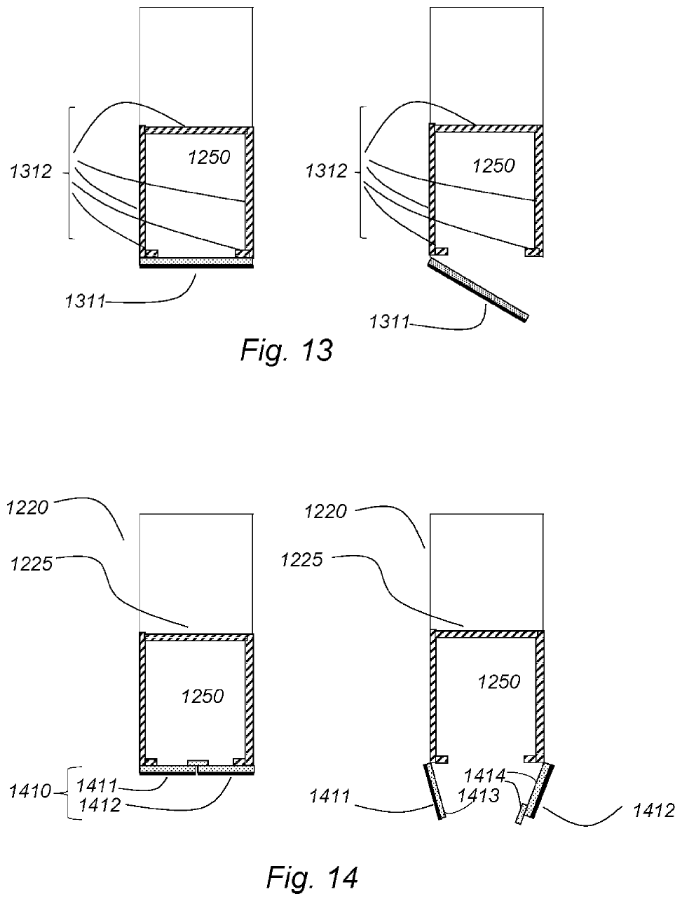

The cabin includes at least one door which could be opened and closed to provide access to the magnetically insulated area. Such door is preferably the rear door of the ambulance. As shown in FIG. 13, in a schematic top cross-sectional view of ambulance 1220, the moving part of the (rear) door is denoted as 1311, and is magnetically shielded with its magnetic shielding shown as a dotted pattern. The left drawing in FIG. 13 corresponds to the situation when the door is closed while the right drawing in FIG. 13 corresponds to the situation when the door is partially opened. While the door is closed, the magnetic shielding of part 1311 overlaps partially with the fixed part of magnetic shielding of the cabin which is denoted as 1312 (shown as striped pattern). Preferably a non magnetic mechanical clump mechanically attaches the two magnetic shields 1312, 1311 in the overlapping area. It is important to note that the view in FIG. 13 is a top view and thus shows only a top cross section, but magnetic shielding should preferably coat the whole area of cabin walls, rear door, interior barrier, top to bottom as well as the whole floor and ceiling of the cabin.

A similar method could also be used when the door is composed of more than one moving part as shown in FIG. 14 which shows a top cross-sectional view of ambulance 1220, similar to FIG. 13, only for the case of a multicomponent door. The left drawing in FIG. 14 corresponds to the situation when the door is closed, while the right drawing in FIG. 14 corresponds to the situation when the door is partially opened. In such a case the (rear) door 1410 is composed of two moving parts 1411 and 1412 which are magnetically shielded by magnetic shields (denoted as dotted pattern) 1413 and 1414, respectively. Magnetic shield 1414 is slightly wider than magnetic shield 1413 and overlaps partially with magnetic shield 1413 from the inside of the ambulance, when parts 1411 and 1412 are closed. It should be noted however that part 1412 should be closed prior to the closing of part 1411 in the case shown. Again a non magnetic mechanical clump is used to attach magnetic shields 1413 and 1414.

It is of preference that the magnetic field generated in the proximity of magnetic shielding by MRI device 1210, will not exceed the saturation field of the magnetic shielding material. The magnetic field of MRI device 1210 should also preferably be small in the proximity of magnetic shielding to avoid deterioration of homogeneity of magnetic field in the imaging volume. It is thus of preference to locate the MRI device away from the magnetically insulating cabin walls. The magnetically insulating cabin walls should preferably be beyond at least the 5 gauss line, and preferably the magnetic field in the proximity of magnetic shielding should be less than 0.5 gauss.

It will be appreciated that the magnetic shielding on the ambulance is preferable but an MRI may be performed also without such magnetic shielding. The necessity of magnetic shielding and its amount is determined by the level of electromagnetic disturbances in the vicinity of the MRI. The shielding maybe also in any amount. While operating in outdoor conditions such as in an ambulance, shielding is preferable as lots of electromagnetic sources (such as nearby cars, the ambulance's own mechanical components, power lines etc) may deteriorate the quality of MRI and thus, may require a substantial amount of shielding compared to indoor environment.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art.

1. A magnet array for MRI apparatus, comprising:
multiple magnet elements made of a permanent magnet material dispersed around a longitudinal axis which passes through an inner predefined imaging volume of the MRI system, wherein at least one group of magnet elements forms a ring coaxial with the longitudinal axis, wherein at least one magnet element of the ring possesses cylindrical symmetry with respect to its own axis of symmetry, wherein the axis of symmetry of the magnet element has a finite component in a direction tangential to the peripheral shape of the ring. The multiple magnet elements are configured to jointly generate a magnetic field of at least a given level of uniformity inside the inner imaging volume; and a frame, which is configured to fixedly hold the multiple magnet rings in place.

2. The magnet array according to claim 1, wherein the magnet elements form multiple magnet rings coaxial with the longitudinal axis.

3. The magnet array according to claim 1, wherein the magnet elements are configured to jointly minimize a fringe field outside the magnet array.

4. The magnet array according to claim 1, wherein each magnet ring has a rotational symmetry with respect to an in-plane rotation of the ring around the longitudinal axis.

5. The magnet array according to claim 1, wherein each element in the at least one group of magnet elements possesses cylindrical symmetry with respect to its own axis of symmetry, wherein the axis of symmetry of the element has a finite component in a direction tangential to the peripheral shape of the ring which the group of elements forms.

6. The magnet array according to claim 1, wherein for a given ring each magnet element of the ring possesses cylindrical symmetry with respect to its own axis of symmetry, wherein the axis of symmetry of the magnet element has a finite component in a direction tangential to the peripheral shape of the ring which the group of elements forms.

7. The magnet array according to claim 1, wherein at least some of the magnet elements encircle a predefined imaging volume of the MRI system, wherein the magnet elements are divided into (i) a first assembly characterized by a first minimal inner radius that is smallest among distances of the magnet elements of the first assembly to the longitudinal axis, and (ii) a second assembly positioned alongside the first assembly along the longitudinal axis and characterized by a second minimal inner radius that is smallest among the distances of the magnet elements of the second assembly to the longitudinal axis, wherein the first minimal inner radius of the first assembly is larger than the second minimal inner radius of the second assembly, wherein a center of the imaging volume is located outside the second assembly.

8. (canceled)

9. The magnet array according to claim 1, wherein the magnet elements are arranged with reflectional asymmetry with respect to the longitudinal axis.

10-12. (canceled)

13. The magnet array according to claim 1, wherein the magnet array comprises at least one magnet ring that has a finite component of magnetization along the longitudinal axis (z) and a finite component of magnetization along the radial (r) direction.

14. The magnet array according to claim 13, wherein the at least one magnet ring that has a finite component of magnetization along the longitudinal axis (z) and a finite component of magnetization along the radial (r) direction comprises at least one magnet element that possesses cylindrical symmetry with respect to its own axis of symmetry, wherein the axis of symmetry of the magnet element has a finite component in a direction tangential to the peripheral shape of the ring.

15. (canceled)

16. A method for producing a magnet array, the method comprising:
positioning multiple magnet elements made of a permanent magnet material around a longitudinal axis which passes through an inner predefined imaging volume of the MRI system, wherein at least one group of magnet elements forms a ring coaxial with the longitudinal axis, wherein at least one magnet element of the ring possesses cylindrical symmetry with respect to its axis of symmetry, wherein the axis of symmetry of the magnet element has a finite component in a direction tangential to the peripheral shape of the ring. The multiple magnet elements are configured to jointly generate a magnetic field of at least a given level of uniformity inside the inner imaging volume; and a frame, which is configured to fixedly hold the multiple magnet rings in place.

17. The method according to claim 16, wherein the magnet elements form multiple magnet rings coaxial with the longitudinal axis.

18. The method according to claim 16, wherein the magnet elements are configured to jointly minimize a fringe field outside the magnet array.

19. The method according to claim 16, wherein each magnet ring has a rotational symmetry with respect to an in-plane rotation of the ring around the longitudinal axis.

20. The method according to claim 16, wherein each element in the at least one group of magnet elements possesses cylindrical symmetry with respect to its own axis of symmetry, wherein the axis of symmetry of the element has a finite component in a direction tangential to the peripheral shape of the ring which the group of elements forms.

21. The method according to claim 16, wherein for a given ring each magnet element of the ring possesses cylindrical symmetry with respect to its own axis of symmetry, wherein the axis of symmetry of the magnet element has a finite component in a direction tangential to the peripheral shape of the ring which the group of elements forms.

22. The method according to claim 16, wherein at least some of the magnet elements encircle a predefined imaging volume of the MRI system, wherein the magnet elements are divided into (i) a first assembly characterized by a first minimal inner radius that is smallest among distances of the magnet elements of the first assembly to the longitudinal axis, and (ii) a second assembly positioned alongside the first assembly along the longitudinal axis and characterized by a second minimal inner radius that is smallest among the distances of the magnet elements of the second assembly to the longitudinal axis, wherein the first minimal inner radius of the first assembly is larger than the second minimal inner radius of the second assembly, wherein a center of the imaging volume is located outside the second assembly.

23. (canceled)

24. The method according to claim 16, wherein the magnet elements are arranged with reflectional asymmetry with respect to the longitudinal axis.

25-27. (canceled)

28. The method according to claim 16, wherein the magnet array comprises at least one magnet ring that has a finite component of magnetization along the longitudinal axis (z) and a finite component of magnetization along the radial (r) direction.

29. The method according to claim 28, wherein the at least one magnet ring that has a finite component of magnetization along the longitudinal axis (z) and a finite component of magnetization along the radial (r) direction comprises at least one magnet element that possesses cylindrical symmetry with respect to its own axis of symmetry, wherein the axis of symmetry of the magnet element has a finite component in a direction tangential to the peripheral shape of the ring.

30. (canceled)
